(19) United States
(12) Patent Application Publication
(10) Pub. No.: US 2011/0319427 A1
(43) Pub. Date: Dec. 29, 2011

(54) USE OF 5AHQ AND BORTEZOMIB FOR THE TREATMENT OF HEMATOLOGICAL DISEASES

(75) Inventor: Aaron D. Schimmer, Thornhill (CA)

(73) Assignee: UNIVERSITY HEALTH NETWORK, Toronto, ON (CA)

(21) Appl. No.: 13/203,878

(22) PCT Filed: Mar. 2, 2010
(86) PCT No.: PCT/CA2010/000282

§ 371 Date: Aug. 30, 2011

Related U.S. Application Data
(60) Provisional application No. 61/157,625, filed on Mar. 5, 2009.

(51) Int. Cl. A61K 31/4965; A61P 35/02; A61P 35/00

(52) U.S. Cl. 514/255.06

(57) ABSTRACT

The disclosure provides methods and compositions for treating hematological malignancies. In particular, the disclosure provides methods and compositions for treating leukemia, lymphoma and multiple myeloma using bortezomit and 5AHQ (5-ammo-8-hydroxyqumolme) and related compounds that bind non-competitively to an alpha unit of 2OS proteasome and inhibit proteasome activity Kits and commercial packages are also disclosed

### RELATED APPLICATIONS

This application claims the benefit of 35 USC §119 based on the priority of co-pending U.S. Provisional Patent Application U.S. 61/157,625, filed Mar. 5, 2009, which is being incorporated herein in its entirety by reference.

### FIELD OF THE DISCLOSURE

The disclosure relates to compositions and methods for treating hematological malignancies, particularly leukemia, lymphoma and multiple myeloma in a subject in need thereof.

### BACKGROUND

All of the reported chemical proteasome inhibitors currently under clinical evaluation, such as bortezomib (Velcade) and NPI-0052, bind threonine residues in the active sites of the beta subunits of the 20S proteasome and thereby inhibit the enzymatic activity of the proteasome competitively [1-3]. Of all proteasome inhibitors under evaluation, only bortezomib is approved by the FDA for the treatment of multiple myeloma and mantle cell lymphoma. However, despite its efficacy in these diseases, few patients achieve complete remission with bortezomib alone and most will relapse [4, 5]. Several mechanisms of resistance to bortezomib have been identified including mutation and over-expression of the b5 subunit of the proteasome to which bortezomib binds [6]. Therefore, molecules that inhibit the proteasome through a mechanism distinct from bortezomib could be useful to overcome some forms of resistance to this drug or be used in combination with bortezomib to improve clinical outcome.

### SUMMARY

The small molecule 5-amino-8-hydroxyquinoline (5AHQ) is herein demonstrated to inhibit the proteasome non-competitively and bind the proteasome complex outside of the catalytic site. Moreover, 5AHQ synergized with bortezomib to inhibit the proteasome and induce cell death. Finally, 5AHQ was able to overcome resistance to bortezomib and induce cell death in bortezomib resistant cells.

Thus, 5AHQ is a novel non-competitive proteasome inhibitor that highlights a new strategy to inhibit the proteasome and is a lead for a new class of therapeutics that may enhance responses to bortezomib or overcome bortezomib resistance.

An aspect of the disclosure provides a method of treating a hematological malignancy comprising administering an effective amount of bortezomib and an effective amount of a compound that binds an alpha subunit of the 20S proteasome to a subject in need of such treatment. Another aspect provides use of an effective amount of bortezomib and an effective amount of a compound that binds an alpha subunit of the 20S proteasome to treat a hematological malignancy. A further aspect provides a composition comprising bortezomib and an effective amount of a compound that binds an alpha subunit of the 20S proteasome for treating a hematological malignancy.

In an embodiment, the compound that binds the alpha subunit of the 20S proteasome is 5AHQ.

Another aspect provides a method of treating a hematological malignancy comprising administering an effective amount of 5AHQ and an effective amount of bortezomib to a subject in need of such treatment.

In an embodiment, the hematological malignancy is leukemia, lymphoma or multiple myeloma.

Another aspect relates to a method of inducing cell death in a hematological cancer cell comprising contacting the cell with an effective amount of bortezomib and an effective amount of a compound that binds an alpha subunit of the 20S proteasome.

Yet another aspect provides a method of inhibiting the 26S proteasome or inhibiting NFkappaB comprising contacting the cell with an effective amount of bortezomib and an effective amount of a compound that binds the alpha subunit of the 20S proteasome.

In yet a further aspect, the disclosure comprises a composition comprising an effective amount of bortezomib and a compound that binds an alpha subunit of the 20S proteasome.

In certain embodiments, the compound that binds the alpha subunit of the 20S proteasome is 5AHQ.

Also provided are kits and a commercial package comprising bortezomib; 5AHQ; and optionally instructions for use.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

An embodiment of the disclosure will now be described in relation to the drawings in which:

FIG. 1 is a graph illustrating a chemical screen identifies 5AHQ as a proteasome inhibitor

A) Protein extracts from MDAY-D2 leukemia cells were treated with aliquots of compounds from a focused chemical library (final concentration 5 μM). After incubation, the preferential chymotrypsin-like substrate Suc-LLVY-AMC was added and the rate of free AMC was measured over time as described in Materials and Methods. Data represent the mean±SD maximal rate of proteasomal activity expressed as a percentage of buffer treated cells. *=5AHQ
B) Chemical structure of 5-amino-8-hydroxyquinoline (5AHQ)

FIG. 2 is a series of graphs demonstrating that 5AHQ inhibits the proteasome non-competitively and binds the alpha subunits A) Proteasomes isolated from rabbit reticulocytes were treated with increasing concentrations of 5AHQ or MG132. After incubation, increasing concentration of the fluorogenic substrate Z-Leu-Leu-Glu-AMC were added and the rate of free AMC was measured over time with a florescent spectrophotometric plate reader (excitation=380 nm, emission=460 nm). Data represent the mean±SD. Lineweaver-Burk plots are presented as a fit to a noncompetitive model of inhibition for 5AHQ and a competitive model for MG132. Inserts: Lineweaver-Burk plots are presented demonstrating a poor fit to a competitive model of inhibition for 5AHQ and a noncompetitive model for MG132.

FIG. 3 is a graph demonstrating 5AHQ synergizes with bortezomib and a pictorial illustration showing 5AHQ binds the alpha subunits

A) Proteasomes isolated from rabbit reticulocytes were treated with bortezomib (2.5, 5, and 10 nM) and or 5AHQ (1, 2 and 4 μM). After incubation, cleavage of the fluorogenic substrate Z-Leu-Leu-Glu-AMC was measured as above. Data represent the mean±SD maximal rate of proteasomal activity expressed as a percentage of buffer treated cells.
B) (Left) Structure of the T. acidophilum proteasome [18] highlighting ante- and proteolytic chambers as well as a binding site of 5AHQ established from NMR studies of the half proteasome. The approximate binding sites of bortezomib that is located inside the catalytic chamber are also indicated. (Right) Inside view of the a-ring, highlighting residues that are affected in NMR spectra upon addition of 5AHQ (darkened region).

FIG. 4 is a series of graphs demonstrating that 5AHQ inhibits the proteasome when added to intact cells

A) Myeloma, leukemia, and solid tumor cell lines were treated with increasing concentrations of 5AHQ. Twenty hours after treatment, cells were lysed and the preferential chymotrypsin-like substrate Suc-LLVY-AMC was added. The generation of free AMC was measured over time with a fluorescent spectrophotometric plate reader as described in the Materials and Methods. Data represent the mean±SD maximal rate of proteasomal activity expressed as a percentage of buffer treated cells.
B) LP1 multiple myeloma cells were treated with increasing concentrations of 5AHQ for 20 hours. After 24 hours of incubation, cells were harvested, and total proteins were isolated. The abundance of ubiquitinated proteins and b-actin expression were measured by SDS-PAGE immunoblotting with anti-ubiquitin and anti-b-actin antibodies, respectively.
C) MDAY-D2 cells were treated with increasing concentrations of 5AHQ for 24 hours followed by treatment with TNF-alpha (10 nM) or buffer control for one hour. After incubation, cells were harvested, nuclear proteins extracted, and NFkappaB activity was measured based on the ability of the p50 subunit to bind its DNA consensus sequence using an enzyme-linked immunosorbent assay as described in the Materials and Methods.

FIG. 5 is a series of graphs demonstrating that 5AHQ induces cell death in malignant cells preferentially over normal hematopoietic cells and synergizes with bortezomib.

A) Leukemia, B) myeloma, and C) solid tumor cell lines were treated with increasing concentrations of 5AHQ. Forty-eight hours after treatment, cell viability was measured by the MTS assay. Data represent the mean±SD percent viability compared to buffer treated cells.
D) Primary acute myeloid leukemia (AML) blasts (n=4), CLL (n=5) or normal peripheral blood stem cells (PBSC) (n=3) were obtained from the peripheral blood of consenting patients with AML, CLL or donors of PBSC for allotransplantation, respectively. Mononuclear cells were isolated by Ficoll separation and treated with increasing concentrations of 5AHQ. Forty-eight hours after incubation, cell viability was measured by MTS (AML and PBSC) or AlamarBlue Fluorescence (CLL) assay. Data represent the mean±SD percentage of viable cells compared to buffer treated cells.
E) OCI-AML2 cells were treated with increasing concentrations of 5AHQ and bortezomib. Forty-eight hours after incubation, cell viability was measured by the MTS assay.

FIG. 6 is a series of graphs which demonstrate that 5AHQ delays tumor growth in mouse models of leukemia.

Sublethally irradiated NOD/SCID mice were injected subcutaneously with U937 and K562 leukemia cells or intraperitoneally with MDAY-D2 leukemia cells. Mice were then treated daily by oral gavage with buffer or 5AHQ (50 mg/kg) dissolved and 0.4% Tween® 80 in PBS. Eight (MDAY-D2) or ten (U937 and K562) days after tumor inoculation, mice were sacrificed, the subcutaneous or intraperitoneal tumor excised, and the weight of the tumors measured. The bars represent the median of the population. p values reflect the Mann-Whitney non-parametric test.

FIG. 7 is a series of graphs that demonstrate that 5AHQ inhibits the proteasome when added to cell extracts

Cellular proteins were extracted from myeloma, leukemia, and solid tumor cell and treated with increasing concentrations of 5AHQ for two hours. After incubation, the fluorogenic substrate Suc-LLVY-AMC was added and the rate of free AMC was measured over time as described in Materials and Methods. Data represent the mean±SD maximal rate of proteasomal activity expressed as a percentage of buffer treated cells

FIG. 8 is a series of graphs that demonstrate that 5AHQ inhibits the T. acidophilum proteasome non-competitively

Proteasomes isolated from T. acidophilum were treated with increasing concentrations of 5AHQ. After incubation, increasing concentrations of the fluorogenic substrate Suc-LLVY-AMC were added and the rate of free AMC was measured over time with a florescent spectrophotometric plate reader (excitation=380 nm, emission=460 nm). Data represent the mean±SD. A Lineweaver-Burk plot is presented as a fit to a noncompetitive model of inhibition.

### DETAILED DESCRIPTION OF THE INVENTION

### I. Definitions

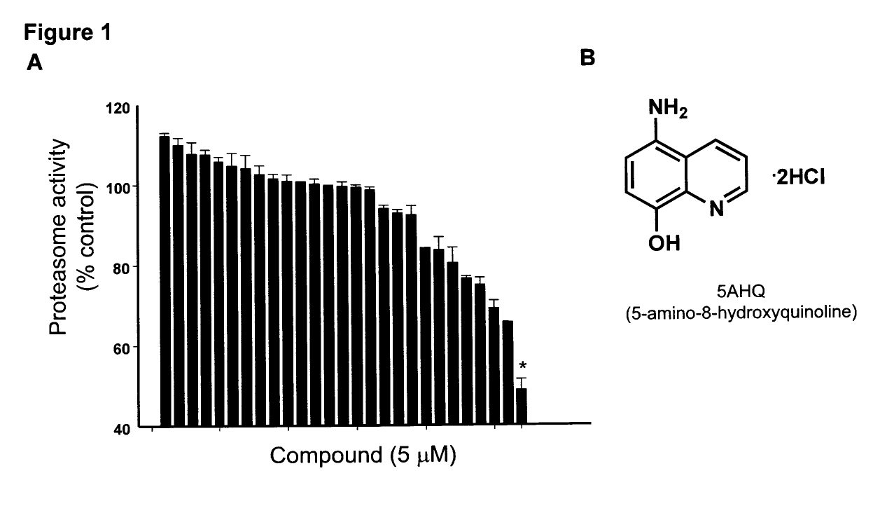

The term “5AHQ” and/or “AHQ” as used herein means 5-amino-8-hydroxyquinoline and includes all pharmaceutically acceptable salts, solvates, and prodrugs thereof as well as combinations thereof. For example, see FIG. 1B.

The term “bortezomib” as used herein refers to a dipeptide boronoic acid analogue having the chemical structure name [(1R)-3-Methyl-1-[[(2S)-1-oxo-3-phenyl-2-[(pyrazinylcarbonyl)amino]propyl]amino]butyl]boronic acid and includes all pharmaceutically acceptable salts, solvates, and prodrugs thereof as well as combinations thereof. Bortezomib is also called PS-341 and is also known by the brand name VELCADE. Bortezomib reversibly and competitively inhibits the 26S proteasome.

The term “cell death” as used herein includes all forms of cell death including necrosis and apoptosis.

The term “pharmaceutically acceptable salt” means an acid addition salt, which is suitable for or compatible with the treatment of patients. The term “pharmaceutically acceptable acid addition salt” as used herein means any non-toxic organic or inorganic salt of any base compound of the disclosure. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the disclosure will vary depending on the compound and the solvate. In general, solvates are formed by dissolving the compound in the appropriate solvent and isolating the solvate by cooling or using an antisolvent. The solvate is typically dried or azeotroped under ambient conditions.

In general, prodrugs will be functional derivatives of the compounds of the disclosure which are readily convertible in vivo into the compound from which it is notionally derived. Prodrugs of the compounds of the disclosure may be conventional esters formed with the available hydroxy and/or amino group. For example, the available OH and/or NH₂ in the compounds of the disclosure may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the disclosure are those in which the hydroxy and/or amino groups in the compounds is masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

As used herein, the phrase “effective amount” or “therapeutically effective amount” means an amount effective, at dosages and for periods of time necessary to achieve the desired result. For example in the context or treating a hematological malignancy, an effective amount is an amount that for example induces remission, reduces tumor burden, and/or prevents tumor spread or growth compared to the response obtained without administration of the compound(s). Effective amounts may vary according to factors such as the disease state, age, sex, weight of the subject. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

As used herein, to “inhibit” or “suppress” or “reduce” a function or activity, such as proteasomal activity or NFkappaB activity, is to reduce the function or activity when compared to otherwise same conditions except for a condition or parameter of interest, or alternatively, as compared to another condition.

As used herein “inhibiting the proteasome” refers to inhibiting one or more proteasome activities for example, chymotrypsin activity and “inhibiting NFkappaB” refers to inhibiting one or more NFkappaB activities for example, transactivation of NKkappaB modulated gene expression or DNA binding.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage multiple myeloma can be treated to prevent progression or alternatively a subject in remission can be treated with a compound or composition described herein to prevent recurrence. Treatment methods comprise administering to a subject a therapeutically effective amount of one or mores compounds described in the present disclosure and optionally consists of a single administration, or alternatively comprises a series of applications. For example, the compounds described herein may be administered at least once a week, from about one time per week to about once daily for a given treatment or the compound may be administered twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

The dosage administered will vary depending on the use and known factors such as the pharmacodynamic characteristics of the particular substance, and its mode and route of administration, age, health, and weight of the individual recipient, nature and extent of symptoms, kind of concurrent treatment, frequency of treatment, and the effect desired. Dosage regime may be adjusted to provide the optimum therapeutic response.

The term “subject” as used herein includes all members of the animal kingdom including mammals, and suitably refers to humans.

The term “hematological malignancy” or “hematologic malignancy” as used herein refers to a cancer that affects blood and bone marrow.

The term “hematological cancer cell” as used herein refers a cancerous cell of the blood and bone marrow lineages, including primary cells. Hematological cancer cells include for example leukemia cells such as leukemia cells represented by THP1, HL-60, RSV411, K562, Jurkat, U937, OCI-M2, OCI-AML2 and NB4 leukemia cell lines and cells phenotypically similar thereto, lymphoma cells such as lymphoma cells represented MDAY-D2 and cell phenotypically similar thereto, and multiple myeloma cells such as multiple myeloma cells represented by OPM2, KMS11, LP1, UTMC2, KSM18 and OCIMy5 myeloma cell lines and cells phenotypically similar thereto. Hematological cancer cells also include chronic myelogenous leukemia cells, including cells representing the blast crises phases such as K562 and cells phenotypically similar thereto; AML cells such as; HL-60, K562, OCI-M2, AND NB4 and cells phenotypically similar thereto, ALL cells such as RSV411 AND JURKAT and cells phenotypically similar thereto, and lymphoma cells such as MDAY-D2 and cells phenotypically similar thereto.

The term “leukemia” as used herein means any disease involving the progressive proliferation of abnormal leukocytes found in hemopoietic tissues, other organs and usually in the blood in increased numbers. For example, leukemia includes acute myeloid leukemia, acute lymphocytic leukemia and chronic myeloma leukemia (CML) in blast crisis.

The term “lymphoma” as used herein means any disease involving the progressive proliferation of abnormal lymphoid cells. For example, lymphoma includes mantle cell lymphoma, Non-Hodgkin's lymphoma, and Hodgkin's lymphoma. Non-Hodgkin's lymphoma would include indolent and aggressive Non-Hodgkin's lymphoma. Aggressive Non-Hodgkin's lymphoma would include intermediate and high grade lymphoma. Indolent Non-Hodgkin's lymphoma would include low grade lymphomas.

The term “myeloma” and/or “multiple myeloma” as used herein means any tumor or cancer composed of cells derived from the hemopoietic tissues of the bone marrow. Multiple myeloma is also knows as MM and/or plasma cell myeloma.

The term “phenotypically similar” refers to a cell type that exhibits morphological, physiological and/or biochemical characteristics similar to another cell type. For example, a cell that is phenotypically similar to an AML cell can include a cell that comprises Auer rods. As another example, U937 cells which are derived from a patient with lymphoma, show morphological similarity to monocytoid AML cells. As a further example the leukemia cell line NB4 differentiates similar to promyelocytic cells (PML) with all trans retinoic acid (ATRA) and thereby represents a model of PML cells.

As used herein, “contemporaneous administration” and “administered contemporaneously” means that two substances are administered to a subject such that they are both biologically active in the subject at the same time. The exact details of the administration will depend on the pharmacokinetics of the two substances in the presence of each other, and can include administering one substance within 24 hours of administration of the other, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances.

As used herein, the term “control” refers to a suitable non-hematological malignancy cell, including, for example cells from an individual or a group of individuals who do not have a leukemic disorder.

The “proteasome” as used herein refers to a multimeric enzymatic complex involved in the degradation of protein and includes a 20S particle and two 19S components which together form the 26S proteasome. The 20S particle consists of four stacked heptameric ring structures that are themselves composed of two different types of subunits; α subunits which are structural in nature, and β subunits which are predominantly catalytic. An example of the human α subunit amino acid sequence is presented in BAG37603. The proteasome comprises multiple protease activities including a chymotrypsin-like protease activity. The proteasomal degradation pathway is necessary to rid cells of excess and misfolded proteins as well as to regulate levels of proteins responsible for processes such as cell cycle progression, DNA repair and transcription.

The term “amino acid corresponding to Ile159, Val113, Val87, Val82, Leu112, Val89, Val134, Val24 and Leu 136 of the T. acidophilum 20S proteasome” as used herein means an amino acid corresponding in terms of position and/or function to the before listed amino acid in any 20S proteasome, for example in the human 20S proteasome. A person skilled in the art would be familiar with methodologies for identifying corresponding amino acids, for example using sequence alignment, structured alignment etc.

The term “proteasomal activity” as used herein refers to an activity of the proteasome.

The term “pharmaceutically acceptable” means compatible with the treatment of animals, in particular, humans.

The term “synergistic” as used herein means the enhanced or magnified effect of a combination on at least one property compared to the individual effects of each component of the combination. For example, compounds that inhibit the proteasome by the same mechanism, e.g binding of a catalytic effect would not be expected to have more than additive effect. Synergism can be assessed and quantified for example by analyzing the Data by the Calcusyn median effect model where the combination index (CI) indicates synergism (CI<0.9), additively (CI=0.9-1.1) or antagonism (CI>1.1). CIs of <0.3, 0.3-0.7, 0.7-0.85, 0.85-0.90, 0.90-1.10 or >1.10 indicate strong synergism, synergism, moderate synergism, slight synergism, additive effect or antagonism, respectively. The CI is the statistical measure of synergy. The FA is the ratio of each drug based on their LD50's.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### II. Methods/Uses

New combination therapeutics for treating hematological malignancies are herein provided.

The proteasome inhibitor bortezomib is approved for the treatment of hematological malignancies, but few patients achieve complete response after treatment with bortezomib alone and most ultimately relapse. Bortezomib and all known chemical proteasome inhibitors currently under clinical evaluation bind the active sites of the proteolytic complex and inhibit the enzymes competitively. To identify proteasome inhibitors that act through different mechanisms, a focused chemical library was screened and the small molecule 5-amino-8-hydroxyquinoline (5AHQ) was identified which inhibited the proteasome non-competitively. NMR studies established that 5AHQ bound the alpha subunits of the 20S proteasome far removed from the catalytic sites of the proteasome. The combination of 5AHQ and bortezomib synergistically inhibited the enzymatic activity of the proteasome. In intact cells, 5AHQ induced cell death in leukemia and myeloma cell lines and primary patient samples preferentially over normal hematopoietic cells. Similar to the effects on the proteasome, the combination of 5AHQ and bortezomib induced cell death synergistically. Consistent with its inhibition of the proteasome through a unique mechanism, 5AHQ remained cytotoxic to bortezomib-resistant THP1 cells with over-expression and mutation of the bortezomib-binding b5 proteasome subunit. Finally, oral administration of 5AHQ delayed tumor growth in mouse models of leukemia without untoward toxicity.

Leukemias, lymphomas, and myelomas are all hematological malignancies. AML cells are generally derived from cells of the myeloid lineage, while other leukemias, lymphomas and myelomas are derived from lymphoid lineage. However, myeloid and lymphoid lineage cells derive from a common hematopoietic precursor. Based on the teachings herein, it is expected that hematological malignancies including leukemias, lymphomas and myelomas are treatable by an effective amount of bortezomib and 5AHQ.

Accordingly, provided herein is a new strategy for the treatment of hematologic malignancies.

An aspect of the disclosure provides a method of treating a hematological malignancy comprising administering an effective amount of bortezomib and an effective amount of a compound that binds an alpha subunit of the 20S proteasome to a subject in need of such treatment. A further aspect provides use of an effective amount of bortezomib and an effective amount of a compound that binds an alpha subunit of the 20S proteasome for treating a hematological malignancy. Yet a further aspect provides an effective amount of bortezomib and an effective amount of a compound that binds an alpha subunit of the 20S proteasome for treating a hematological malignancy. In an embodiment, the bortezomib and compound that binds the alpha subunit of the 20S proteasome are comprised in a composition.

In an embodiment, the compound that binds the alpha subunit of the 20S proteasome induces a spectral change at least one amino acid corresponding to Ile159, Val113, Val87, Val82, Leu112, Val89, Val134, Val24 and Leu 136 of the T. acidophilum 20S proteasome. In an embodiment, the compound that binds the alpha subunit of the 20S proteasome induces a spectral change at least 2, at least 3, at least 4, at least 5, at least 6, at least 7, at least 8 or at each of the amino acids corresponding to Ile159, Val113, Val87, Val82, Leu112, Val89, Val134, Val24 and Leu 136 of the T. acidophilum 20S. In an embodiment, the compound that binds the alpha subunit of the 20S proteasome is 5AHQ.

Another aspect of the disclosure provides a method of treating a hematological malignancy comprising administering an effective amount of 5AHQ and an effective amount of bortezomib to a subject in need of such treatment. Another aspect provides use of an effective amount of 5AHQ and an effective amount of bortezomib for treating a hematological malignancy. A further aspect provides an effective amount of bortezomib and 5AHQ for treating hematological malignancies. In an embodiment, the bortezomib and the 5AHQ are comprised in a composition.

In an embodiment, the hematological malignancy is a leukemia such as acute myeloid leukemia. In another embodiment, the leukemia is acute lymphocytic leukemia. In another embodiment the subject has high-risk acute myeloid leukemia. In another embodiment, the hematological malignancy is multiple myeloma. In another embodiment, the hematological malignancy is lymphoma. In a further embodiment, the lymphoma is mantle cell lymphoma. In another embodiment, the lymphoma is Non-Hodgkin's lymphoma. In another embodiment, the lymphoma is Hodgkin's lymphoma. In a further embodiment, the Non-Hodgkin's lymphoma is selected from indolent and aggressive Non-Hodgkin's lymphoma. In another embodiment, the aggressive Non-Hodgkin's lymphoma is selected from an intermediate and a high grade lymphoma. In another embodiment, the indolent Non-Hodgkin's lymphoma is a low grade lymphoma.

A further aspect of the disclosure provides a method of inducing cell death in a hematological cancer cell comprising contacting the cell with an effective amount of bortezomib and an effective amount of a compound that binds an alpha subunit of the 20S proteasome. Also provided is use of an effective amount of bortezomib and an effective amount of a compound that binds an alpha subunit of the 20S proteasome for inducing cell death in a hematological cancer cell. A further aspect includes an effective amount of bortezomib and an effective amount of a compound that binds an alpha subunit of the 20S proteasome for inducing cell death in a hematological cancer cell. In an embodiment, the compounds are comprised in a composition.

In an embodiment, the compound that binds the 20S proteasome is 5AHQ.

In an embodiment, the hematological cancer cell is a leukemia cell, a lymphoma cell or a myeloma cell.

In one embodiment, the cell is or is phenotypically similar to an acute myeloid leukemia cell. In another embodiment, the cell is or is phenotypically similar to an acute lymphoid leukemia cell. In another embodiment, the cell is phenotypically similar to a chronic myelogenous leukemia cell. In other embodiments, the cell is phenotypically similar to a lymphoma or multiple myeloma cell.

In certain embodiment, the cell is in vivo.

A further aspect of the disclosure provides a method of inhibiting the 26S proteasome comprising contacting the cell with an effective amount of bortezomib and an effective amount of a compound that binds the alpha subunit of the 20S proteasome. Also provided in an embodiment, is use of an effective amount of bortezomib and an effective amount of a compound that binds the alpha subunit of the 20S proteasome for inhibiting the 26S proteasome in a cell. Another embodiment, the disclosure includes an effective amount of bortezomib and an effective amount of a compound that binds the alpha subunit of the 20S proteasome for inhibiting the 26S proteasome in a cell. In and embodiment, the compounds are comprised in a composition.

In an embodiment, the compound that binds the 20S proteasome is 5AHQ.

In an embodiment, the 26S proteasome is inhibited by at least 40%. In another embodiment, the 26S proteasome is inhibited by at least 50%, at least 60%, at least 70%, at least 80%, at least 90% or at least 95% as determined for example using assays described herein and/or known in the art.

A further aspect provides a method of inhibiting NFkappaB in a cell comprising contacting the cell with an effective amount of bortezomib and an effective amount of a compound that binds the alpha subunit of the 20S proteasome. Also provided in an embodiment, is use of an effective amount of bortezomib and an effective amount of a compound that binds the alpha subunit of the 20S proteasome for inhibiting NFkappaB in a cell. Another embodiment, the disclosure includes an effective amount of bortezomib and an effective amount of a compound that binds the alpha subunit of the 20S proteasome for inhibiting NFkappaB in a cell. In and embodiment, the compounds are comprised in a composition.

In an embodiment, the compound that binds the 20S proteasome is 5AHQ.

In an embodiment, the NFkappB is inhibited by at least 40%. In another embodiment, the NFkappB is inhibited by at least 50%, at least 60%, at least 70%, at least 80%, at least 90% or at least 95% as determined for example using assays described herein and/or known in the art.

In another aspect, the disclosure describes methods and uses wherein the bortezomib and 5AHQ are comprised in a composition described herein.

In yet a further aspect, the disclosure describes methods and uses wherein the bortezomib and 5AHQ are comprised in a dosage or dosage form described herein.

The dosage administered will vary depending on the use and known factors such as the pharmacodynamic characteristics of the particular substance, and its mode and route of administration, age, health, and weight of the individual recipient, nature and extent of symptoms, kind of concurrent treatment, frequency of treatment, and the effect desired. Dosage regime may be adjusted to provide the optimum therapeutic response.

In an embodiment, the dosage of bortezomib when administered in combination with 5AHQ is at least 10% less, at least 20%, at least 30%, at least 40%, at least 50%, at least 60% or at least 70% less compared to when bortezomib is administered alone. For example, at least 10% less bortezomib can be used when administered with 5AHQ to achieve the same level of cell death, 26S proteasome inhibition, and/or treatment efficacy.

In an embodiment, bortezomib and 5AHQ are administered or contacted with the cell sequentially. In one embodiment, the bortezomib is administered or contacted with the cell prior to administering or contacting the cell with 5AHQ. In another embodiment, 5AHQ is administered or contacted with the cell prior to administering or contacting the cell with bortezomib. In a further embodiment, the bortezomib and the 5AHQ are administered contemporaneously.

As mentioned, bortezomib and 5AHQ, as are demonstrated herein, interact synergistically. In an embodiment, the effective amount of bortezomib and the effective amount of 5AHQ is sufficient for a combination index of at least 0.8, at least 0.7, at least 0.6, at least 0.5 or at least 0.4.

A person skilled in the art will recognize that the methods and uses described herein can also be combined with other therapies known in the art.

### III. Compositions

Another aspect of the disclosure provides a composition comprising an effective amount of bortezomib and a compound that binds an alpha subunit of the 20S proteasome. An aspect provides a composition comprising an effective amount of bortezomib and a compound that binds an alpha subunit of the 20S proteasome for treating hematological malignancies. In an embodiment, the composition is a pharmaceutical composition. In an embodiment, the compound that binds the alpha subunit of the 20S proteasome is 5AHQ.

It is to be clear that the present disclosure describes pharmaceutically acceptable salts, solvates and prodrugs of bortezomib and 5AHQ and mixtures comprising two or more of bortezomib and 5AHQ, pharmaceutically acceptable salts of bortezomib and 5AHQ, pharmaceutically acceptable solvates of bortezomib and 5AHQ and prodrugs of bortezomib and 5AHQ.

Where the compounds described herein possess more than one asymmetric centre, they may exist as diastereomers. It is to be understood that all such isomers and mixtures thereof in any proportion are encompassed within the scope of the present disclosure. It is to be understood that while the stereochemistry of the compounds of the application may be as provided for in any given compound listed herein, such compounds of the disclosure may also contain certain amounts (e.g. less than 20%, suitably less than 10%, more suitably less than 5%) of compounds of the disclosure having alternate stereochemistry.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In an embodiment, the composition comprises the pharmaceutically acceptable salt of 5AHQ, 5-amino-8-hydroxyquinoline hydrochloride. In another embodiment, the 5AHQ is 5-amino-8-hydroxyquinoline dihydrochloride.

Compositions include 5AHQ and bortezomib prodrugs. In general, such prodrugs will be functional derivatives of 5AHQ and bortezomib which are readily convertible in vivo into the compound from which it is notionally derived. Prodrugs of 5AHQ and bortezomib may be conventional esters formed with the available hydroxy. For example, the available OH in 5AHQ or bortezomib may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the disclosure are those in which one or more of the hydroxy groups in the compounds are masked as groups which can be converted to hydroxy groups in vivo. Conventional procedures for the selection and preparation of suitable prodrugs are described, for example, in “Design of Prodrugs” ed. H. Bundgaard, Elsevier, 1985.

5AHQ and an effective amount of bortezomib are suitably formulated into pharmaceutical compositions for administration to human subjects in a biologically compatible form suitable for administration in vivo. Accordingly, an embodiment further includes a pharmaceutical composition comprising 5AHQ and an effective amount of bortezomib and a pharmaceutically acceptable carrier and/or diluent.

The disclosure in one aspect, also describes a pharmaceutical composition comprising an effective amount of 5AHQ and an effective amount of bortezomib and a pharmaceutically acceptable carrier and/or diluent for treatment of a hematological malignancy, optionally leukemia, lymphoma or multiple myeloma in a subject in need of such treatment.

The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences. On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween®), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound, together with a suitable amount of carrier so as to provide the form for direct administration to the patient.

The compositions described herein can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer. Wherein the route of administration is oral, the dosage form may be for example, incorporated with excipient and used in the form of enteric coated tablets, caplets, gelcaps, capsules, ingestible tablets, buccal tablets, troches, elixirs, suspensions, syrups, wafers, and the like. The dosage form may be solid or liquid.

Accordingly in one embodiment, the disclosure describes a pharmaceutical composition wherein the dosage form is a solid dosage form. A solid dosage form refers to individually coated tablets, capsules, granules or other non-liquid dosage forms suitable for oral administration. It is to be understood that the solid dosage form includes, but is not limited to, non-controlled release, controlled release and time-controlled release dosage form units, employed suitably in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet.

Compositions suitable for buccal or sublingual administration include tablets, lozenges, and pastilles, wherein the active ingredient is formulated with a carrier such as sugar, acacia, tragacanth, or gelatin and glycerin.

In another embodiment, the disclosure describes a pharmaceutical composition wherein the dosage form is a liquid dosage form. A liquid dosage form refers to non-solid dosage forms suitable for, but not limited to, intravenous, subcutaneous, intramuscular, or intraperitoneal administration. Solutions of 5AHQ and bortezomib described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999.

Sustained or direct release compositions can be formulated, e.g. liposomes or those wherein the active compound is protected with differentially degradable coatings, such as by microencapsulation, multiple coatings, etc. It is also possible to freeze-dry the compounds of the disclosure and use the lyophilates obtained, for example, for the preparation of products for injection.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

In an embodiment, the dosage form comprises about 20 mg to about 5000 mg and an effective amount of bortezomib. In another embodiment, the dosage form comprises about 20 mg to about 2500 mg of 5AHQ and an effective amount of bortezomib. In another embodiment, the dosage form comprises about 100 mg to about 2500 mg of 5AHQ and an effective amount of bortezomib. The dosage form may alternatively comprise about 250 mg to about 2500 mg, about 500 mg to about 2500 mg, about 750 mg to about 2500 mg or about 1000 mg to about 2500 mg of 5AHQ and an effective amount of bortezomib. In a further embodiment, the dosage form comprises about 1000 mg of 5AHQ and an effective amount of bortezomib. In other alternative embodiments, the dosage form comprises about 1 to about 200 mg of 5AHQ/kg body weight, about 5 to about 50 mg of 5AHQ/kg body weight, about 10 to about 40 mg of 5AHQ/kg body weight or about 25 mg of 5AHQ/kg body weight of a subject in need of such treatment.

A further aspect is a composition, wherein the oral dosage form is selected from enteric coated tablets, caplets, gelcaps, and capsules, comprising from about 20 mg to about 5000 mg, about 20 mg to about 2500 mg, about 100 mg to about 2500 mg, about 250 mg to about 2500 mg, about 500 mg to about 2500 mg, about 750 mg to about 2500 mg or about 1000 mg to about 2500 mg, suitably from about 50 to about 1500 mg, of 5AHQ and an effective amount of bortezomib. In an embodiment, the oral dosage form is selected from enteric coated tablets, caplets, gelcaps, and capsules, comprising from about 500 mg or about 1000 mg of 5AHQ and an effective amount of bortezomib.

An effective amount of bortezomib includes for example, amounts approved by the FDA. For example, bortezomib is optionally administered as a bolus IV injection 1× or 2× per week at about 1.3 mg/m². In an embodiment, the effective amount of bortezomib is at least 0.5 mg/m², at least 0.6 mg/m², at least 0.7 mg/m², at least 0.8 mg/m², at least 0.9 mg/m², at least 1.0 mg/m², at least 1.1 mg/m², at least 1.2 mg/m², and/or at least 1.3 mg/m². In an embodiment, the dosage regimen is for example about 0.5-1.3 mg/m² by bolus injection 1-2× weekly, 2 out of 3 weeks, for up to 24 weeks. In another embodiment, the dosage regimen is for example about 0.5-1.3 mg/m² by bolus injection on days 1, 4, 8, and 11 on a 28 day cycle for 24 weeks. In another embodiment, the dosage regimen is for example about 0.5-1.3 mg/m² on days 1, 4, 8, 11, 22, 25, 29, and 32 of a 6 week cycle during cycles 1 to 4 and on days 1, 8, 22, and 29 of a 6 week cycle during cycles 5 to 9.

### IV. Kits and Commercial Packages

Another aspect provides a commercial package comprising bortezomib and an effective amount of a compound that binds an alpha subunit of the 20S proteasome. In an embodiment, the compound that binds the alpha subunit is 5AHQ. In another embodiment, the commercial package comprises a composition described herein, and associated therewith instructions for the use thereof for treatment of a hematological malignancy such as acute myeloid leukemia or acute lymphoid leukemia in a subject in need of such treatment. In another embodiment, the commercial package comprises a composition for the treatment of chronic myelogenous leukemia, lymphoma or multiple myeloma. Another embodiment provides a commercial package comprising a composition described herein, and associated therewith instructions for the inducing cell death and/or inhibiting proteasome activity in a hematological cancer cell.

Another aspect of the disclosure provides a kit. In an embodiment, the kit comprises bortezomib and an effective amount of a compound that binds an alpha subunit of the 20S proteasome. In an embodiment the compound that binds the alpha subunit is 5AHQ. In another embodiment, the kit comprises bortezomib, 5AHQ and instructions for use.

The following non-limiting examples are illustrative of the present disclosure:

### EXAMPLES

### Example 1

### Materials and Methods

Cell lines. Human multiple myeloma (MM) and solid tumor cell lines were grown in Iscove's Modified Dulbecco's Medium (IMDM). Human and murine leukemia cell lines were maintained in RPMI-1640 medium. Human myelomonocytic THP1/WT cells and the bortezomib resistant mutants THP1/BTZ50, THP1/BTZ100, THP1/BTZ200 and THP1/BTZ500 were grown in the presence of 50, 100, 200, and 500 nM of bortezomib (Millennium Pharmaceutics, Cambridge, USA)), respectively and RPMI-1640 supplemented with 20 mM HEPES and 2 mM glutamine [6]. These cells were maintained in bortezomib up to at least 7 days prior to the experiment. Media were supplemented with 10% fetal calf serum (FCS), 100 ug/mL penicillin, and 100 units/mL streptomycin (all from Hyclone, Logan, Utah).

Primary cells. Primary human acute myeloid leukemia (AML) and chronic lymphocytic leukemia (CLL) samples were isolated from peripheral blood samples from consenting patients with AML and CLL, respectively, who had at least 80% malignant cells among the mononuclear cells in their peripheral blood. Primary normal hematopoietic cells were obtained from healthy volunteers donating peripheral blood mononuclear stem cells (PBSC) for allotransplantation. Mononuclear cells were isolated by Ficoll density centrifugation. Primary cells were cultured at 37° C. in IMDM supplemented with 10% FCS, 1 mM L-glutamine and appropriate antibiotics. The collection and use of human tissue for this study was approved by the local ethics review board (University Health Network, Toronto, ON, Canada).

### Assessment of the Enzymatic Activity of the Proteasome.

To assess the effects of 5AHQ on the proteasome when added to cell lysates, cellular proteins were extracted from myeloma, leukemia and solid tumor cells with lysis buffer (50 mM HEPES pH 7.5, 150 mM NaCl, 1% Triton X-100, and mM ATP). Equal protein amounts were treated with increasing concentrations of 5AHQ in assay buffer (50 mM Tris-HCl, pH 7.5, 150 mM NaCl). After incubation, the proteasome substrate Suc-LLVY-AMC was added and the rate of free AMC was measured over time with a fluorescent spectrophotometric plate reader (excitation=380 nm, emission=460 nm).

To measure the effects of 5AHQ on the enzymatic activity of the proteasome in intact cells, solid tumor, leukemia and myeloma cell lines were treated with increasing concentrations of 5AHQ. Twenty hours after treatment, cells were lysed in 50 mM HEPES pH 7.5, 150 mM NaCl and 1% Triton X-100, and 2 mM ATP. To equal protein concentrations, the proteasome fluorogenic substrate, Suc-LLVY-AMC, was added and the generation of free AMC was measured over time with a fluorescent spectrophotometric plate reader as above.

The effects of 5AHQ on the enzymatic activity of purified proteasome were also assessed. Proteasome complexes were isolated from rabbit reticulocytes and T. acidophilum as described previously [7, 8]. Increasing concentrations of 5AHQ, MG132, and bortezomib were added to isolated rabbit proteasome in assay buffer (50 mM Hepes, Ph 7.5, 1 mM DTT, and 0.018% SDS) as previously described [9] and to purified T. acidophilum in assay buffer (50 mM Tris-HCl, pH 7.5, 150 mM NaCl) as previously described [8]. After one hour of incubation at 37° C., AMC-conjugated proteasomat substrates were added and the rate of release of free AMC was measured over time with a fluorescent spectrophotometric plate reader as above.

### Assessment of Inhibitor Binding to the Proteasome by NMR.

The 20S proteasome from T. acidophilum was produced as described previously [7] with the alpha and beta subunits co-expressed from 2 different plasmids (where the beta subunit carried a C terminal His-tag). Samples of the half-proteasome comprised of pairs of alpha-rings, a₇-a₇, were produced by overexpression of the a subunit that carried a N-terminal cleavable His-tag that was removed during purification. Methyl labelling of proteins for NMR analysis was achieved by growing cells in D₂O based minimal medium with ²H-¹²C glucose as the carbon source. One hour before induction of protein expression, alpha-ketobutyric acid (60 mg/L) and alpha-ketoisovaleric acid (100 mg/L; only one of the two isopropyl methyls was ¹³CH₃ labeled, the other ¹²CD₃) were added to introduce ¹³CH₃ methyl groups in Ile, Leu and Val [10]. Proteins were purified by Ni-NTA affinity resin and size exclusion chromatography.

NMR experiments with the (a₇-a₇) proteasome were performed on Varian 600 MHz spectrometers at 50° C. on samples that were ˜4 mM in proteasome (˜50 mM in monomer), 100% D₂O, 25 mM potassium phosphate pH 6.8, 50 mM NaCl, 1 mM EDTA, 0.03% NaN₃ and 2 mM DTT similar to previously described [7, 11]. NMR data was processed and analyzed with the nmrPipe/nmrDraw software package [12].

### Cell Viability, and Apoptosis Assays.

Cell viability was assessed by the MTS (Promega, Madison, Wis.) and Alamar Blue Fluorescence assays (Invitrogen, Carlsbad, Calif.) according to manufacturer's instructions and as previously described [13] [14]. Apoptosis was measured by Annexin V-FITC (Biovision Research Products, Mountain View, Calif.) staining and flow cytometry according to manufacturer's instructions and as previously described [15].

### Assessment of 5AHQ's Anti-Leukemic Activity In Vivo.

MDAY-D2 murine leukemia cells (5×10⁵) were injected intraperitoneally into sublethally irradiated (3.5 Gy) NOD/SCID mice (Ontario Cancer Institute, Toronto, Canada). Mice were then treated with 5AHQ (Sigma, St. Louis, Mo.) at 50 mg/kg/day in 0.4% Tween® 80 in PBS or vehicle control by oral gavage for 8 days. Mice were sacrificed, and the weight and volume of the intraperitoneal tumors were measured.

K562 leukemia cells (3×10⁶) or U937 leukemia cells (2×10⁶) were injected subcutaneously (s.c.) into the flank of sublethally irradiated NOD/SCID mice. When tumors where palpable, mice were treated with 50 mg/kg of 5AHQ daily in PBS with 0.4% Tween® 80 or vehicle control by oral gavage daily for 10 days. Mice were then sacrificed, and the weight of the tumors was measured.

Animal studies were carried out according to the regulations of the Canadian Council on Animal Care and with the approval of the local ethics review board.

### Immunoblotting.

Whole cell lysates were prepared from myeloma cells as described previously [13]. Briefly, cells were washed with phosphate-buffered saline (PBS, pH 7.4) and re-suspended in lysis buffer [10 mM Tris (pH 7.4), 150 mM NaCl, 0.1% Triton X-100, 0.5% sodium deoxycholate, and 5 mM EDTA] containing protease inhibitors (Complete tablets, Roche, Indianapolis, Ind.). Protein concentrations were determined by the Bradford assay. Equal amounts of protein were subjected to SDS-PAGE gels followed by transfer to nitrocellulose membranes. Membranes were probed with rabbit anti-human polyclonal anti-ubiquitin (1:1000 v/v) (Calbiochem, San Diego, Calif.), or mouse monoclonal anti-beta actin (1:10,000 v/v) (Sigma) followed by secondary horseradish peroxidase (HRP)-conjugated goat anti-mouse (1:10,000 v/v) or anti-rabbit IgG (1:5,000 v/v) (Amersham Bioscience UK, Little Chalfont, England). Detection was performed by the Enhanced Chemical Luminescence (ECL) method (Pierce, Rockford, Ill.).

### NFkappaB Activity.

NFkappaB activity was measured using the Trans-AM NFkappaB p65 transcription factor assay kit (Active Motif, Carlsbad, Calif.) according to the manufacturer's instructions. Briefly, MDAY-D2 cells were treated with increasing concentrations of 5AHQ for 24 hours. Cells were then treated with TNF-alpha (10 nM) or buffer control for one hour before harvesting. Nuclear extracts were prepared using the Nuclear Extract kit (Active Motif, Carlsbad, Calif.) according to the manufacturer's instructions. Binding of the NFkappaB transcription factor subunit p50 to the DNA consensus sequence (5′-GGGACTTTCC-3′) cross linked to 96-well plates was measured using enzyme-linked immunosorbent assay (ELISA) technology with absorbance reading according to the manufacturer's protocol.

### Statistical Analysis.

Data are expressed as mean values±SD. The statistical significance was computed with Student's t-test. A p value of <0.05 was considered statistically significant. The kinetics by which 5AHQ and MG132 inhibited rabbit muscle purified proteasome were determined using SigmaPlot software (Systat Software, San Jose, Calif.). Isobologram analysis was performed with CalcuSyn software (Biosoft, Ferguson, Mo., and Cambridge, UK). A combination index (CI) less than 1.0 indicated synergism [16].

### Results

### 5AHQ Inhibits the Proteasome Non-Competitively

To identify novel inhibitors of the proteasome a focused chemical library was screened to identify compounds which inhibited the enzymatic activity of the proteasome when added to protein extracts from malignant cells. This screen, identified 5-amino-8-hydroxyquinoline 5AHQ as the most potent inhibitor (FIG. 1A,B). When added to extracts derived from MDAY-D2 leukemia cells, 5AHQ inhibited the enzymatic activity of the proteasome at low micromolar concentrations. Similar effects of 5AHQ were observed when the compound was added to extracts from a panel of myeloma, leukemia and solid tumor cell lines (FIG. 7). Of note, 5AHQ appeared more active in extracts derived from myeloma and leukemia cell lines than extracts from solid tumor cell lines.

### 5AHQ Inhibits the Proteasome Non-Competitively

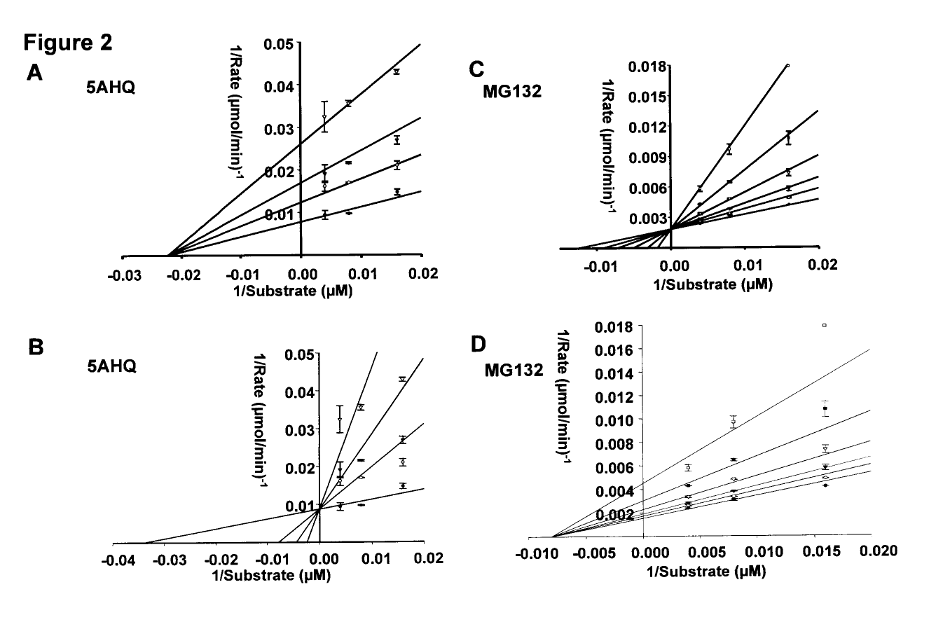

To investigate the mechanism by which 5AHQ inhibited the proteasome, detailed enzymatic studies were conducted using purified proteasomes isolated from rabbit reticulocytes. By Lineweaver-Burk plot analysis, 5AHQ inhibited the rabbit proteasome noncompetitively with a Ki of 2.1±0.18 μM, (FIG. 2A). Notably, the enzyme activity vs. inhibitor concentration did not fit well to competitive or uncompetitive models of inhibition (FIG. 2B). Similar to the effects on the rabbit proteasome, 5AHQ also inhibited purified proteasome isolated from archaebacterium T. acidophilum with non-competitive kinetics (FIG. 8). However, it was a less potent inhibitor with a Ki of 161±4 μM, possibly reflecting small differences in the binding pocket of the inhibitor, although both proteasomes share a similar quaternary structure [17, 18]. In contrast to the non-competitive inhibition observed with 5AHQ, MG132 which binds the active site of the proteasome [8] inhibited the rabbit proteasome competitively, (FIG. 2). Thus, 5AHQ inhibits the proteasome through a mechanism distinct from MG132.

### The Combination of 5AHQ and Bortezomib Inhibit the Proteasome Synergistically

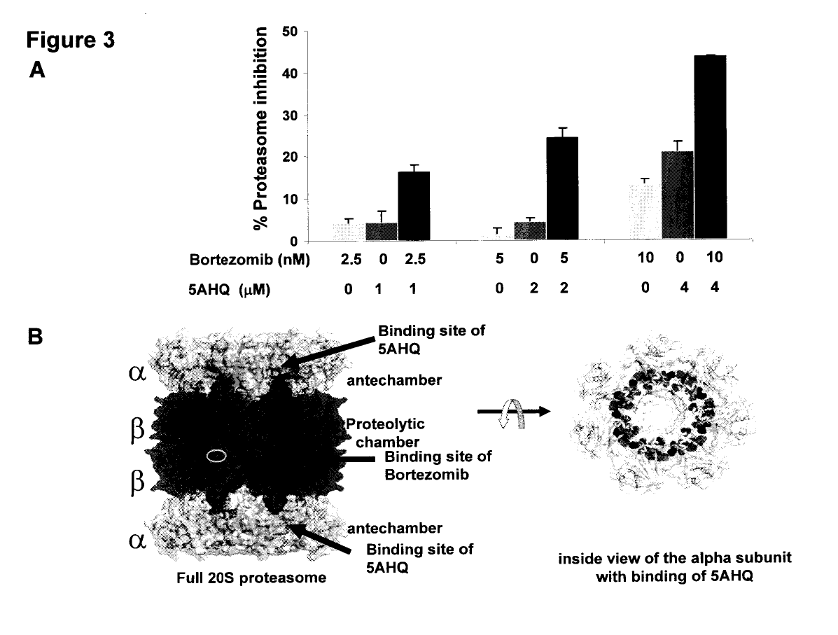

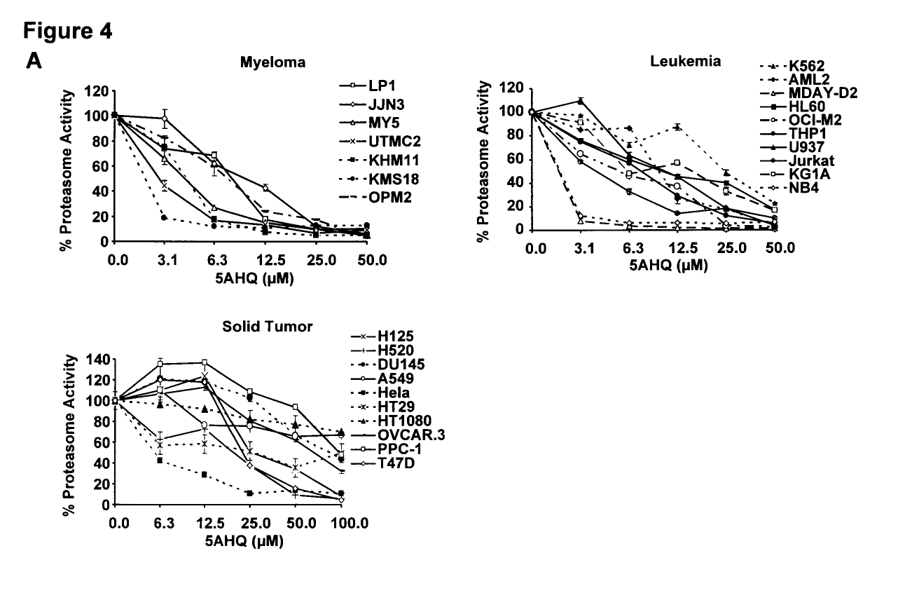

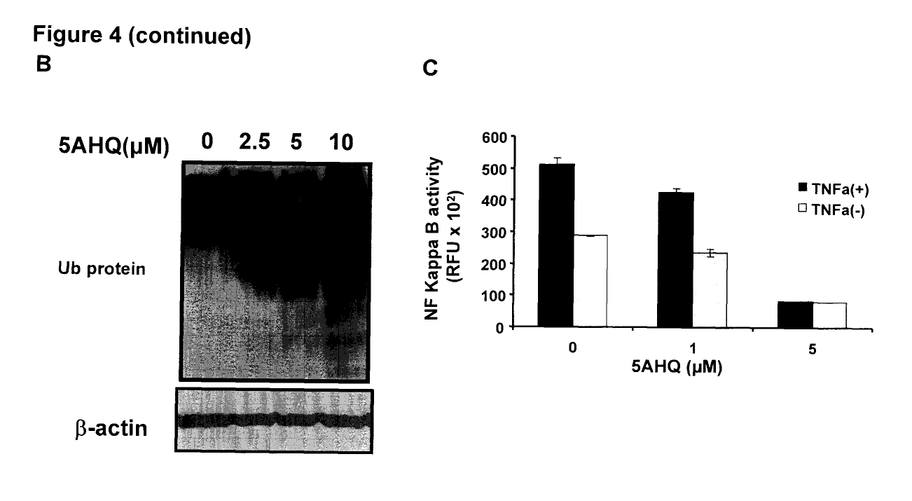

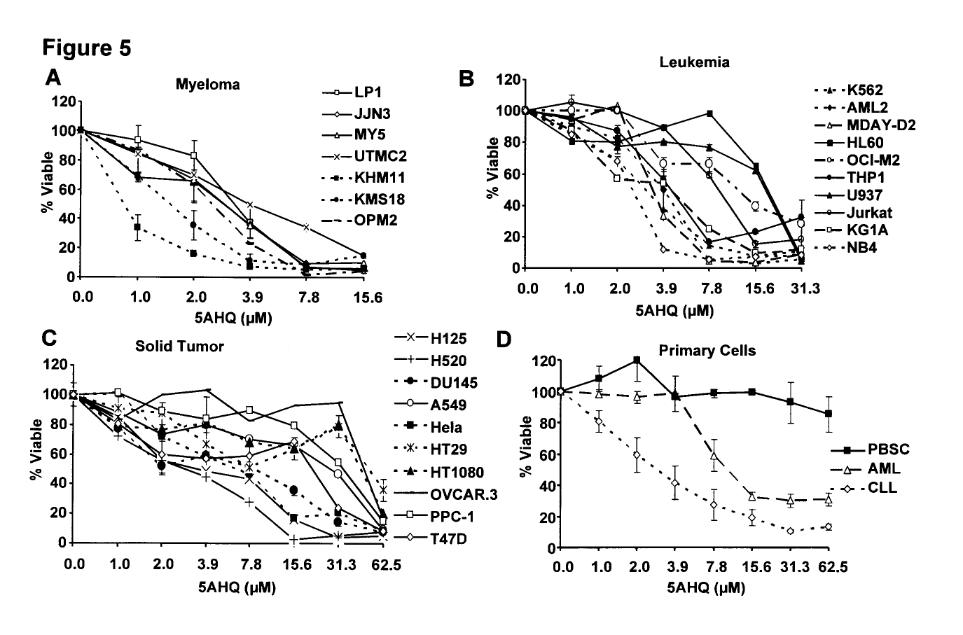

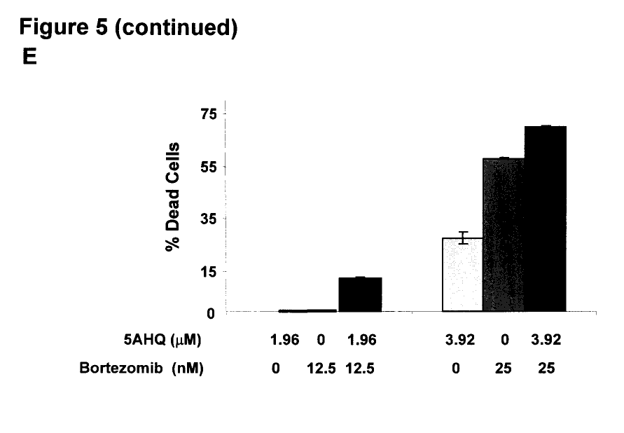

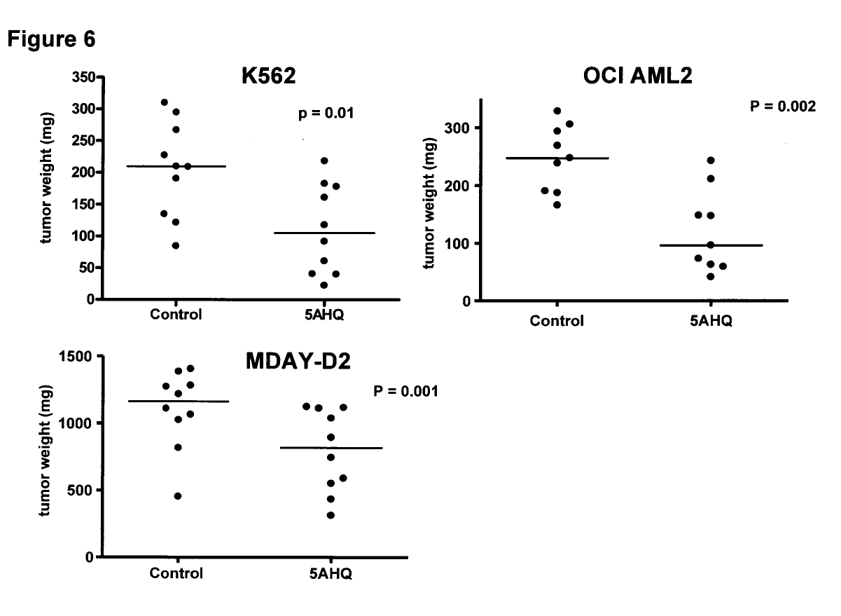

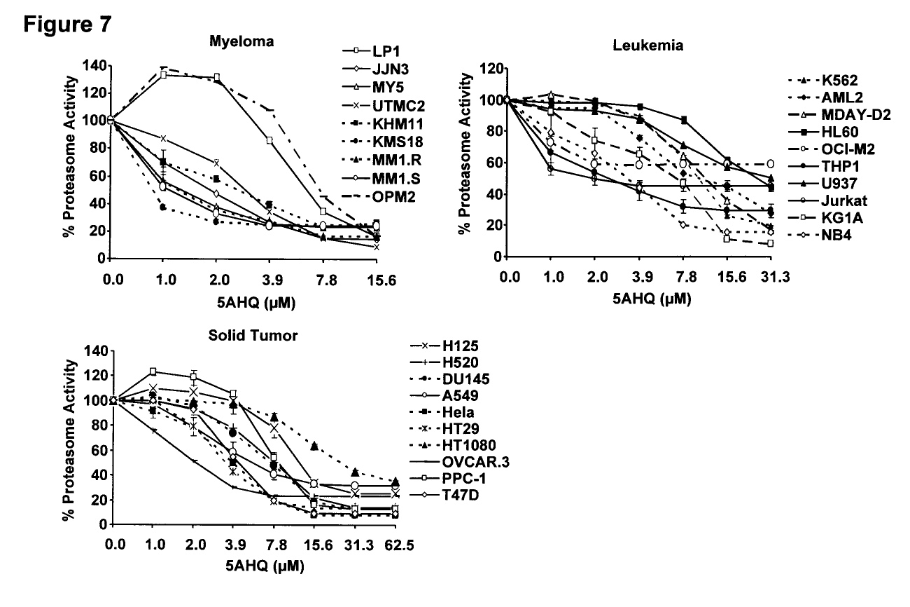

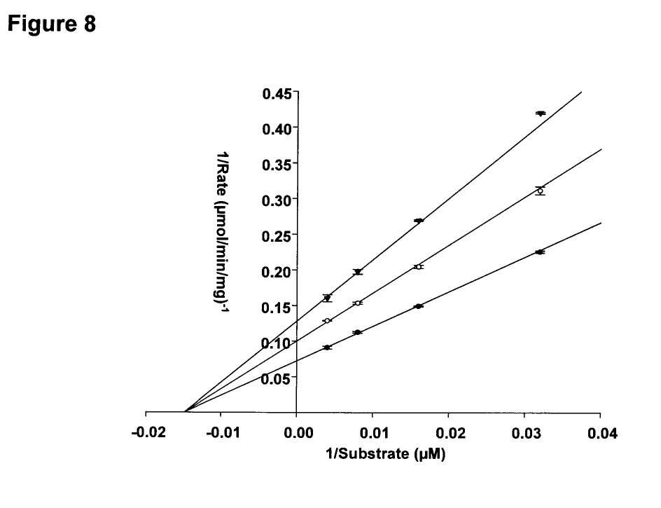

Since 5AHQ inhibits the proteasome through a mechanism distinct from competitive proteasome inhibitors such as MG132 and bortezomib [3], the combination of 5AHQ and bortezomib on the enzymatic activity of the proteasome was computed. 5AHQ synergized with bortezomib and produced greater proteasome inhibition than either agent alone with CI values of 0.46, 0.63, and 0.86 at the Fa 10, Fa 25, and Fa 50, respectively, where a CI value of <1 is synergistic (FIG. 3A).

### 5AHQ Binds the Alpha Subunits of the 20S Proteasome

Given that 5AHQ inhibits the proteasome through a unique mechanism, its interaction with this complex was evaluated by NMR. Initial NMR studies probing the interaction between 5AHQ and full length T. acidophilum proteasome were not successful since the elevated temperatures (65° C.) and significant recording times necessary to obtain high quality NMR spectra (12-16 hours at a proteasome concentration of ˜4 μM used here) of this 670 kDa complex lead to degradation of drug. By contrast, NMR spectra of a smaller half-proteasome construct comprised of a pair of α-rings[7], α₇-α₇, (molecular weight of 360 kDa) can be recorded rapidly and are of high quality even at 50° C. Therefore, the interactions of 5AHQ with α₇-α₇ were evaluated.

The interaction of 5AHQ with α₇-α₇ produced clear spectral changes localized to residues Ile159, Val113, Val87, Val82, Leu112, Val89, Val134, Val24 and Leu136 which are inside the antechamber (FIG. 3B). In contrast, MG132 which binds the proteolytic chamber produced shifts in the beta rings of the full proteasome, as expected and previously described [8]. Thus, 5AHQ binds the antechamber of the proteasome at a site distinct from the substrate binding region. However, the possibility that 5AHQ also binds to sites on the beta-subunits cannot be excluded.

### 5AHQ Inhibits the Proteasome in Intact Cells

Given the ability of 5AHQ to inhibit the enzymatic activity of the proteasome in cell-free assays, the effects of 5AHQ on proteasome function in intact cells was assessed. Leukemia, myeloma, and solid tumor cell lines were treated with increasing concentration of 5AHQ for 24 hours. After treatment, cells were harvested, lysed, and the chymotrypsin-like activity of the proteasome was measured by monitoring the rate of cleavage of the fluorescent substrate Suc-LLVY-AMC. 5AHQ inhibited the rate of Suc-LLVY-AMC cleavage in the malignant cell lines similar to its effects when added to cell extracts (FIG. 4A).

To further assess the effects of 5AHQ on the function of the proteasome in intact cells, LP1 myeloma cell lines were treated with increasing concentrations of 5AHQ. Twenty four hours after treatment, the abundance of ubiquitinated proteins in the cells was measured by immunoblotting. At low micromolar concentrations, 5AHQ increased the amount of ubiquitinated protein, consistent with inhibition of the proteasome (FIG. 4B).

Inhibition of the proteasome with compounds such as bortezomib inhibits signalling through the NFkappaB pathway. Therefore, NFkappaB signalling after treatment of cells with 5AHQ was evaluated. MDAY-D2 cells were treated with 5AHQ with and without TNF-alpha to stimulate NFkappaB signalling. After 24 hours of incubation, DNA binding of the NFkappaB p50 subunit was measured by DNA-binding ELISA. 5AHQ inhibited basal and TNF-alpha stimulated NFkappaB activity at concentrations associated with its enzymatic inhibition of the proteasome (FIG. 4C).

### 5AHQ Induces Cell Death in Malignant Cells Preferentially Over Normal Cell

Inhibition of the proteasome can induce cell death in malignant cells. Therefore, the effects of 5AHQ on cell viability both in cell lines and primary patient samples was assessed. Leukemia, myeloma and solid tumor cell lines were treated with increasing concentrations of 5AHQ for 72 hours and cell viability was measured by the MTS assay. 5AHQ induced cell death with an LD50<5 μM in 7/7 myeloma, 6/10 leukemia, and 3/10 solid tumor cell lines. Cell death was confirmed by Annexin V staining. Cell death induced by 5AHQ matched its ability to inhibit the proteasome (FIG. 5A-C).

The effects of 5AHQ on the viability of primary AML, CLL, and normal hematopoietic cells was also tested. AML and CLL samples were obtained from the peripheral blood of consenting patients and normal hematopoietic cells obtained from peripheral blood of volunteer donors of peripheral blood stem cells for allogeneic blood and marrow transplant. Primary cells were treated with 5AHQ for 72 hours and cell viability was measured. 5AHQ induced cell death in primary AML and CLL cells with an LD50 in the low micromolar range. In contrast, it was not cytotoxic to normal hematopoietic cells up to 62.5 μM (FIG. 5D).

5AHQ Synergizes with Bortezomib

As the combination of 5AHQ and bortezomib synergistically inhibited the proteasome, the cytotoxicity of this combination was also evaluated. 5AHQ synergized with bortezomib to induce cell death that was greater than either compound alone, with CI values of 0.31, 0.48, and 0.74, at the Fa 10, Fa 25, and Fa 50, respectively (FIG. 5E).

### 5AHQ Overcomes Bortezomib Resistance

The efficacy of bortezomib is hampered by the emergence of drug resistance due, in part, to over-expression and mutation of the bortezomib-binding 35 proteasome subunit [6] or over-expression of multidrug resistant pumps [19]. As 5AHQ inhibits the proteasome through a mechanism distinct from bortezomib, 5AHQ was evaluated in bortezomib-resistant cells. THP1/BTZ50, THP1/BTZ100, THP1/BTZ100, THP1/BTZ500 leukemia cells in which the β5 proteasome subunit is mutated and over-expressed [6] were treated with increasing concentrations of 5AHQ or bortezomib for 72 hours. After incubation, cell viability was measured by trypan blue staining. THP1 cells with up to 237-fold resistance to bortezomib remained essentially fully sensitive to 5AHQ (Table 1). In these bortezomib-resistant cell lines, the addition of bortezomib to 5AHQ neither enhanced nor antagonized the activity of 5AHQ.

The activity of 5AHQ in CEM cells resistant to bortezomib due to over-expression the multidrug resistant pumps Pgp, BCRP, or MRP1 was also explored. These bortezomib resistant cells also remained equally sensitive to 5AHQ-induced cell death. Thus, 5AHQ can overcome some forms of bortezomib resistance.

### 5AHQ Delays Tumor Growth in Xenograft Models

As 5AHQ induced cell death in malignant cells, the effects of oral 5AHQ in 3 mouse models of leukemia were evaluated. Sublethally irradiated NOD-SCID mice were injected subcutaneously with OCI-AML2 or K562 human leukemia cells or intraperitoneally with MDAY-D2 murine leukemia cells. After tumor implantation, mice were treated with 5AHQ (50 mg/kg/day) or buffer control by oral gavage. Oral 5AHQ decreased tumor weight and volume in all 3 mouse models by at least 50% compared to control without causing weight loss or gross organ toxicity (FIG. 6). Of note the LD50 of 5AHQ in mice was >1000 mg/kg.

TABLE 1
Growth inhibitor effects of 5AHQ against human leukemia cells with
acquired resistance to Bortezomib (BTZ)

| Cell Line | IC₅₀ 5AHQ (μM) | RF 5AHQ | RF BTZ^{#} |
|---|---|---|---|
| THP1/WT | 3.7 ± 0.3 | 1 | 1 |
| THP1/BTZ50 | | | |
| (−) | 6.6 ± 0.4 | 1.8 | 45 |
| (+) | 5.9 ± 0.4 | 1.6 | |
| THP1/BTZ100 | | | |
| (−) | 6.3 ± 0.4 | 1.7 | 79 |
| (+) | 6.2 ± 0.6 | 1.7 | |
| THP1/BTZ200 | | | |
| (−) | 6.4 ± 0.7 | 1.7 | 129 |
| (+) | 6.2 ± 0.4 | 1.7 | |
| THP1/BTZ500 | | | |
| (−) | 6.6 ± 0.9 | 1.8 | 237 |
| (+) | 5.3 ± 0.8 | 1.4 | |

(−) experiment conducted with cells grown in absence of BTZ for at least 3 days
(+) experiment conducted with cells grown in presence of BTZ
Results represent the mean + SD of 3 separate experiments
RF = Resistance Factor (IC50 resistant cell line/IC50 parental cell line)
^{#}Data from Oerlemans et al, 2008 [6]

### Discussion

Proteasome inhibitors improve the clinical outcome of patients with multiple myeloma and mantle cell lymphoma and are currently being evaluated for the treatment of other malignancies including leukemia [4, 5, 20]. However bortezomib and all of the other chemical proteasome inhibitors currently under clinical evaluation block the complex competitively by binding the active sites of the enzymes [1-3]. The activity of 5AHQ is demonstrated herein to be a unique proteasome inhibitor that blocks the enzyme complex non-competitively. NMR studies establish that this compound, binds the alpha subunits of the antechamber of the 20S proteasome, a novel binding site that is distinct from the binding site of bortezomib. Although the possibility that 5AHQ simultaneously binds to 1′-subunits cannot be excluded, the kinetic assays support a mechanism of inhibition that is different than binding to the active sites. The exact binding site of 5AHQ to the proteasome can be determined using methods known to the person skilled in the art, such as NMR or crystallography methods.

As 5AHQ and bortezomib inhibited the proteasome through distinct mechanisms, the combination of these agents was evaluated. The combination of 5AHQ and bortezomib inhibited the proteasome and induced cell death synergistically. Thus, combining competitive and non-competitive proteasome inhibitors is a novel strategy to increase response rates to proteasome inhibition. Alternatively, if the side effect profiles of these two agents are distinct, the combination of these two agents could permit the use of lower doses of bortezomib which would reduce the incidence and severity of bortezomib toxicity.

Several mechanisms of bortezomib resistance have been identified including increased levels of heat shock protein 27 [21] and increased expression of multidrug resistance pumps [19]. Recently, acquired mutation or over-expression of the β5 proteasome has also been demonstrated to confer resistance to bortezomib [6]. Consistent with a binding a site on the proteasome distinct from bortezomib, 5AHQ retained full activity in cell lines with mutated or increased β5 subunits. Moreover, 5AHQ retained activity in cells over-expressing multidrug resistant pumps. Therefore, 5AHQ overcomes some forms of bortezomib resistance. Thus, these findings support the development of this class of compound for some patients who relapse after bortezomib treatment.

Currently, it is unknown how 5AHQ inhibits the proteasome non-competitively, but one possibility is that binding outside of the active site produces a conformational change that prevents substrates from entering the proteolytic chamber of the complex. Previously, a 39 amino acid peptide PR39 was shown to bind the 20S proteasome and inhibit the enzyme noncompetitively [22]. This peptide is postulated to bind to a site distal from the catalytic region and function via an allosteric mechanism of action whereby binding leads to changes in proteasome structure. However, characterization of the binding sites of PR39 on the proteasome has yet to be reported. Chloroquine, also inhibits the proteasome non-competitively by binding a similar but not overlapping region of the alpha subunits in the T. acidophilum proteasome [8]. However, chloroquine is a much weaker proteasome inhibitor than 5AHQ and the concentrations of chloroquine required to inhibit the proteasome in intact mammalian cells are not pharmacologically achievable in animals or humans. In the future, detailed crystal structures of the 5AHQ-proteasome interaction will better discern this mechanism.

5AHQ induced cell death at concentrations associated with its ability to inhibit the proteasome and block NFkappaB signalling. These results suggest that the cytotoxicity of 5AHQ is related to its effects on the proteasome. However, it cannot be excluded that 5AHQ has additional targets beyond the proteasome that may also contribute to its anti-cancer effects.

In summary 5AHQ is a novel inhibitor of the proteasome that synergizes with and overcomes resistance to the competitive proteasome inhibitor, bortezomib. As such, it demonstrates a new strategy for inhibition of the proteasome and a potential lead for a new class of therapeutic agents.

### Example 2

### Xenograft Model

Sublethally irradiated NOD/SCID mice will be injected subcutaneously with OCI-AML2 leukemia or LP1 myeloma cells. After tumor implantation, mice will be treated with bortezomib intraperitoneally, 5AHQ orally or intraperitoneally, and a combination of the two agents. Tumor size will be measured over time. Gross toxicity and body weight will also be measured over time.

Combination with Bortezomib In Vitro and In Vivo:

The combination of bortezomib with the synthetic derivative of retinoic acid, fenretinide, has been shown to induce cell death in cultured melanoma cells synergistically. In vivo, the combination of these two agents delayed tumor growth in a mouse model of melanoma more than either agent alone [23]. Likewise, the combination of bortezomib with the BcI-2 inhibitor ABT737 [24] or the polyamine analogue, CGC-11093 [25] also synergized in vitro and in vivo. This supports that in vitro synergistic effects seen with bortezomib and 5AHQ are expected to be seen in vivo.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent disclosures are herein incorporated by reference in their entirety to the same extent as if each individual publication, patent or patent disclosure was specifically and individually indicated to be incorporated by reference in its entirety.

### FULL CITATIONS FOR REFERENCES REFERRED TO IN THE SPECIFICATION

1. Williamson, M. J., Blank, J. L., Bruzzese, F. J., Cao, Y., Daniels, J. S., Dick, L. R., Labutti, J., Mazzola, A. M., Patil, A. D., Reimer, C. L., Solomon, M. S., Stirling, M., Tian, Y., Tsu, C. A., Weatherhead, G. S., Zhang, J. X., and Rolfe, M. (2006). Comparison of biochemical and biological effects of ML858 (salinosporamide A) and bortezomib. Mol Cancer Ther 5, 3052-3061.
2. Groll, M., Huber, R., and Potts, B. C. (2006). Crystal structures of Salinosporamide A (NPI-0052) and B (NPI-0047) in complex with the 20S proteasome reveal important consequences of beta-lactone ring opening and a mechanism for irreversible binding. J Am Chem Soc 128, 5136-5141.
3. Groll, M., Berkers, C. R., Ploegh, H. L., and Ovaa, H. (2006). Crystal structure of the boronic acid-based proteasome inhibitor bortezomib in complex with the yeast 20S proteasome. Structure 14, 451-456.
4. Richardson, P. G., Sonneveld, P., Schuster, M. W., Irwin, D., Stadtmauer, E. A., Facon, T., Harousseau, J. L., Ben-Yehuda, D., Lonial, S., Goldschmidt, H., Reece, D., San-Miguel, J. F., Blade, J., Boccadoro, M., Cavenagh, J., Dalton, W. S., Boral, A. L., Esseltine, D. L., Porter, J. B., Schenkein, D., and Anderson, K. C. (2005). bortezomib or high-dose dexamethasone for relapsed multiple myeloma. N Engl J Med 352, 2487-2498.
5. Fisher, R. I., Bernstein, S. H., Kahl, B. S., Djulbegovic, B., Robertson, M. J., de Vos, S., Epner, E., Krishnan, A., Leonard, J. P., Lonial, S., Stadtmauer, E. A., O'Connor, O. A., Shi, H., Boral, A. L., and Goy, A. (2006). Multicenter phase II study of bortezomib in patients with relapsed or refractory mantle cell lymphoma. J Clin Oncol 24, 4867-4874.
6. Oerlemans, R., Franke, N. E., Assaraf, Y. G., Cloos, J., van Zantwijk, I., Berkers, C. R., Scheffer, G. L., Debipersad, K., Vojtekova, K., Lemos, C., van der Heijden, J. W., Ylstra, B., Peters, G. J., Kaspers, G. L., Dijkmans, B. A., Scheper, R. J., and Jansen, G. (2008). Molecular basis of bortezomib resistance: proteasome subunit beta5 (PSMB5) gene mutation and overexpression of PSMB5 protein. Blood 112, 2489-2499.
7. Sprangers, R., and Kay, L. E. (2007). Quantitative dynamics and binding studies of the 20S proteasome by NMR. Nature 445, 618-622.
8. Sprangers, R., Li, X., Mao, X., Rubinstein, J. L., Schimmer, A. D., and Kay, L. E. (2008). TROSY-based NMR evidence for a novel class of 20S proteasome inhibitors. Biochemistry 47, 6727-6734.
9. Kisselev, A. F., Akopian, T. N., Woo, K. M., and Goldberg, A. L. (1999). The sizes of peptides generated from protein by mammalian 26 and 20S proteasomes. Implications for understanding the degradative mechanism and antigen presentation. J Biol Chem 274, 3363-3371.
10. Goto, N. K., Gardner, K. H., Mueller, G. A., Willis, R. C., and Kay, L. E. (1999). A robust and cost-effective method for the production of Val, Leu, Ile (delta 1) methyl-protonated 15N-, 13C-, 2H-labeled proteins. J Biomol NMR 13, 369-374.
11. Tugarinov, V., Hwang, P. M., Ollerenshaw, J. E., and Kay, L. E. (2003). Cross-correlated relaxation enhanced 1H[bond]¹³C NMR spectroscopy of methyl groups in very high molecular weight proteins and protein complexes. J Am Chem Soc 125, 10420-10428.
12. Delaglio, F., Grzesiek, S., Vuister, G. W., Zhu, G., Pfeifer, J., and Bax, A. (1995). NMRPipe: a multidimensional spectral processing system based on UNIX pipes. J Biomol NMR 6, 277-293.
13. Mawji, I. A., Simpson, C. D., Hurren, R., Gronda, M., Williams, M. A., Filmus, J., Jonkman, J., Da Costa, R., Wilson, B., Thomas, M. P., Reed, J. C., Glinsky, G., and Schimmer, A. D. (2007). Critical role for Fas-associated death domain-like interleukin-1-converting enzyme-like inhibitory protein in anoikis resistance and distant tumor formation. JNCI 99, 811-822.
14. Al-Nasiry, S., Geusens, N., Hanssens, M., Luyten, C., and Pijnenborg, R. (2007). The use of Alamar Blue assay for quantitative analysis of viability, migration and invasion of choriocarcinoma cells. Hum Reprod 22, 1304-1309.
15. Carter, B. Z., Gronda, M., Wang, Z., Welsh, K., Pinilla, C., Andreeff, M., Schober, W. D., Nefzi, A., Pond, G. R., Mawji, I. A., Houghten, R. A., Ostresh, J., Brandwein, J., Minden, M. D., Schuh, A. C., Wells, R. A., Messner, H., Chun, K., Reed, J. C., and Schimmer, A. D. (2005). Small-molecule XIAP inhibitors derepress downstream effector caspases and induce apoptosis of acute myeloid leukemia cells. Blood 105, 4043-4050.
16. Chou, T. C., and Talalay, P. (1984). Quantitative analysis of dose-effect relationships: the combined effects of multiple drugs or enzyme inhibitors. Adv Enzyme Regul 22, 27-55.
17. Zwickl, P., Lottspeich, F., Dahlmann, B., and Baumeister, W. (1991). Cloning and sequencing of the gene encoding the large (alpha-) subunit of the proteasome from Thermoplasma acidophilum. FEBS Lett 278, 217-221.
18. Lowe, J., Stock, D., Jap, B., Zwickl, P., Baumeister, W., and Huber, R. (1995). Crystal structure of the 20S proteasome from the archaeon T. acidophilum at 3.4 A resolution. Science 268, 533-539.
19. Minderman, H., Zhou, Y., O'Loughlin, K. L., and Baer, M. R. (2007). bortezomib activity and in vitro interactions with anthracyclines and cytarabine in acute myeloid leukemia cells are independent of multidrug resistance mechanisms and p53 status. Cancer Chemother Pharmacol 60, 245-255.
20. Codes, J., Thomas, D., Koller, C., Giles, F., Estey, E., Faderl, S., Garcia-Manero, G., McConkey, D., Ruiz, S. L., Guerciolini, R., Wright, J., and Kantarjian, H. (2004). Phase I study of bortezomib in refractory or relapsed acute leukemias. Clin Cancer Res 10, 3371-3376.
21. Chauhan, D., Li, G., Shringarpure, R., Podar, K., Ohtake, Y., Hideshima, T., and Anderson, K. C. (2003). Blockade of Hsp27 overcomes bortezomib/proteasome inhibitor PS-341 resistance in lymphoma cells. Cancer Res 63, 6174-6177.
22. Gaczynska, M., Osmulski, P. A., Gao, Y., Post, M. J., and Simons, M. (2003). Proline- and arginine-rich peptides constitute a novel class of allosteric inhibitors of proteasome activity. Biochemistry 42, 8663-8670.
23. Hill D S, Martin S, Armstrong J L, Flockhart R, Tonison J J, Simpson D G, Birch-Machin M A, Redfern C P, Lovat P E. Combining the endoplasmic reticulum stress-inducing agents bortezomib and fenretinide as a novel therapeutic strategy for metastatic melanoma. Clin Cancer Res. 2009 Feb. 15; 15(4):1192-8.
24. Paoluzzi L, Gonen M, Bhagat G, Furman R R, Gardner J R, Scotto L, Gueorguiev V D, Heaney M L, Manova K, O'Connor O A. The BH3-only mimetic ABT-737 synergizes the antineoplastic activity of proteasome inhibitors in lymphoid malignancies. Blood. 2008 Oct. 1; 112(7):2906-16. Epub 2008 Jun. 30.
25. Carew J S, Nawrocki S T, Reddy V K, Bush D, Rehg J E, Goodwin A, Houghton J A, Casero R A Jr, Marton L J, Cleveland J L.; The novel polyamine analogue CGC-11093 enhances the antimyeloma activity of bortezomib. Cancer Res. 2008 Jun. 15; 68(12):4783-90.

1. A method of treating a hematological malignancy comprising administering an effective amount of bortezomib and an effective amount of a compound that binds an alpha subunit of the 20S proteasome to a subject in need of such treatment.

2. The method of claim 1, wherein the compound that binds the alpha subunit of the 20S proteasome induces a spectral change at least one amino acid corresponding to Ile159, Val113, Val87, Val82, Leu112, Val89, Val134, Val24 and Leu 136 of the T. acidophilum 20S proteasome.

3. The method of claim 1, wherein the compound that binds the alpha subunit of the 20S proteasome is 5AHQ.

4. The method of claim 3 for treating a hematological malignancy comprising administering an effective amount of 5AHQ and an effective amount of bortezomib to a subject in need of such treatment.

5. The method of claim 1 wherein the hematological malignancy is a leukemia, lymphoma or multiple myeloma.

6. The method of claim 5 wherein the leukemia is acute myeloid leukemia, acute lymphocytic leukemia, high-risk acute myeloid leukemia or wherein the lymphoma is mantle cell lymphoma, Non-Hodgkin's lymphoma, Hodgkin's lymphoma, indolent Non-Hodgkin's lymphoma, aggressive Non-Hodgkin's lymphoma.

7. (canceled)

8. The method of claim 1 for inducing cell death in a hematological cancer cell comprising contacting the cell with an effective amount of bortezomib and an effective amount of a compound that binds an alpha subunit of the 20S proteasome.

9. The method of claim 8, wherein the compound that binds the 20S proteasome is 5AHQ.

10. The method of claim 8, wherein the hematological cancer cell is a leukemia cell, a lymphoma cell or a myeloma cell.

11. The method of claim 8, wherein the cell is in vivo.

12. The method of claim 1 for inhibiting the 26S proteasome and/or NFkappaB comprising contacting the cell with an effective amount of bortezomib and an effective amount of a compound that binds the alpha subunit of the 20S proteasome.

13. The method of claim 12 wherein the compound that binds the alpha subunit of the 20S proteasome is 5AHQ and the 26S proteasome and/or NFkappaB is inhibited by at least 40%.

14. The method of claim 4, wherein the 5AHQ and the bortezomib are administered sequentially or contemporaneously.

15. The method of claim 4, wherein the effective amount of bortezomib and the effective amount of 5AHQ is sufficient for a combination index of at least 0.8.

16. A composition comprising an effective amount of bortezomib and a compound that binds an alpha subunit of the 20S proteasome.

17. The composition of claim 16, wherein the composition is a pharmaceutical composition.

18. The composition of claim 16, wherein the compound that binds the alpha subunit of the 20S proteasome is 5AHQ.

19. The composition of claim 18, wherein the 5AHQ is 5-amino-8-hydroxyquinoline hydrochloride or 5-amino-8-hydroxyquinoline dihydrochloride.

20. The composition of claim 18, wherein the bortezomib is suitable for administration by injection and the 5AHQ is suitable for oral administration.

21-39. (canceled)

40. A kit or commercial package comprising:
a. bortezomib;
b. 5AHQ; and optionally
c. instructions for use.

41. The kit or commercial package wherein the bortezomib is suitable for administration by injection and/or the 5AHQ is suitable for oral administration.
